(19) United States
(12) Patent Application Publication
(10) Pub. No.: US 2009/0081703 A1
(43) Pub. Date: Mar. 26, 2009

(54) METHOD FOR DETECTING DEEP VENOUS THROMBOSIS

(76) Inventor: Steven H. Yale, Marshfield, WI (US)

Correspondence Address: BOYLE FREDRICKSON S.C., 840 North Plankinton Avenue, MILWAUKEE, WI, 53203, US

(21) Appl. No.: 12/238,011

(22) Filed: Sep. 25, 2008

Related U.S. Application Data
(60) Provisional application No. 60/975,043, filed on Sep. 25, 2007.

(51) Int. Cl. G01N 33/573

(52) U.S. Cl. 435/7.4

(57) ABSTRACT

A method for detecting deep vein thrombosis (DVT) in humans includes the steps of assaying a collected blood sample and measuring the amount of Lp-PLA₂. The detected amount of Lp-PLA₂ is then compared and correlated with known risk values of Lp-PLA₂. A diagnosis of a patient's likelihood, risk, or development of DVT may be made by comparing the measured value with values known to correlate to a particular risk or development of DVT.

### CROSS REFERENCE TO RELATED APPLICATIONS

The present application claims the benefit of U.S. Provisional Patent Application Ser. No. 60/975,043 filed on Sep. 25, 2007.

### BACKGROUND OF THE INVENTION

Thrombosis is the formation of a blood clot, i.e., a thrombus, inside a blood vessel. Thrombi are capable of obstructing the flow of blood through the vessels throughout the circulatory system. In a relatively large vessel, the blood flow may simply be decreased whereas when the thrombus occurs in a relatively small vessel, blood flow may be completely obstructed and in some cases may result in the death of the tissue supplied by the vessel.

When a thrombus occurs within the deep veins, i.e., those veins that are deep within the body, as opposed to superficial veins which are close to the surface of the skin, it is called a deep venous thrombosis or DVT. DVT typically affect the leg veins such as the femoral vein, popliteal vein, or the deep veins of the pelvis. Common symptoms of DVT include pain, swelling and redness of the affected area as well as dilation of the surface veins.

There are several known risk factors that contribute to the development of DVT including age, obesity, infection, immobilization, contraception usage, tobacco usage, and air travel. These risk factors are often categorized in one of three different groups known to affect clot formation. Specifically, thrombus development is typically associated with at least one of the following: (1) rate of flow through the vessel, (2) the consistency or thickness of the blood flowing through the vessel and (3) the quality of the vessel wall. The most common causes of DVT, however, are recent surgery and hospitalization.

DVT may be diagnosed through a variety of means including physical examination, imaging, and by performing blood tests for markers commonly associated with DVT. In a physical examination, DVT may be diagnosed by measuring the circumference of the affected contralateral limb at a particular point and palpating the venous tract. Typically, however, physical diagnoses are often unreliable for excluding a diagnosis of DVT. The most reliable method of diagnosing DVT is intravenous venography which involves injecting a peripheral vein of an affected limb with a contrast agent and taking X-rays to determine whether the venous supply has become obstructed. Although imaging techniques such as X-ray are the best known ways to diagnose DVT, blood tests may be utilized for testing for markers commonly associated with the development of DVT. For example, thrombin-antithrombin-complex (TAT) and fibrin/fibrinogen-degration product (FDP)-D-dimer are presently used as markers for DVT diagnoses.

If a thrombus becomes dislodged and free floating within the circulatory system it is an embolus. Emboli are cable of migrating through a person's circulatory system and causing a blockage or occlusion of the vessel in another part of the body similar to a thrombus. The free-floating embolus may propagate through the circulatory system potentially resulting in embolization. For example, the embolization may affect the pulmonary vasculature if the embolus floats to and cuts off circulation thereto. As such, the pulmonary vasculature may become occluded and result in a pulmonary infarction or pulmonary embolism.

DVT and pulmonary embolisms are manifestations of the same clinical condition, namely, venous thromboembolism (VTE). VTE is the pathophysiological process of thrombus formation wherein red blood cells, fibrin, and to a lesser extent platelets and leukocytes form a mass within the cardiovascular system.

The development of VTE may be primary, also known as idiopathic, or secondary. In the case of idiopathic VTE, the development of VTE is unprovoked or unassociated with any known risk factor. The development of VTE may be considered secondary when associated with at least one known risk factor. It is estimated that 145 per 100,000 persons in the general population develop symptomatic DVT of which 69 per 100,000 persons experience a pulmonary embolism. VTE disease remains a significant cause of mortality and morbidity despite widespread availability of effective prophylactic regimes in hospitalized patients.

Presently there are no known methods of predicting the development of idiopathic VTE. Further, there are no known methods of predicting the recurrence of DVT in patients undergoing anticoagulant therapy for the treatment of DVT.

### SUMMARY OF THE INVENTION

The present invention predicts that Lp-PLA₂ is elevated in patients with acute VTE disease and that the levels of Lp-PLA₂ in patients with atherosclerotic disease are useful in modeling and predicting the risk of venous thrombosis and recurrent VTE events.

Higher levels of Lp-PLA₂ are known to be linked with an increased incidence of atherosclerotic disease. The present inventors predict that higher concentrations of Lp-PLA₂ may be linked to higher incidence of idiopathic VTE. The present inventors theorize that patients with idiopathic causes for DVT have higher levels of Lp-PLA₂s compared to those with secondary causes for VTE.

The present invention further predicts that elevated Lp-PLA₂ levels contribute to the pathogenesis of idiopathic VTE through its effect in promoting an inflammatory response in atherosclerotic plaques.

Thus, it is an object of the present invention to provide a method for predicting idiopathic VTE.

In another embodiment, the present invention provides that elevated Lp-PLA₂ levels are an indication for predicting risk for recurrent VTE. For example, monitoring levels of Lp-PLA₂ at the time that drugs such as Warfarin or Coumadin is discontinued may also be useful in medical decision making to determine whether patients with higher levels should remain on anticoagulation due to increased risk for recurrent VTE.

It is yet another object of the present invention to provide a method of providing healthcare providers with useful medical information for determining the appropriate time for the cessation of anticoagulants in patients with VTE having an increased risk of recurrent VTE.

### DETAILED DESCRIPTION OF THE INVENTION

The present invention proposes a method of predicting occurrences of idiopathic DVT. Thrombus formations in the arterial and venous sides of the circulation have traditionally been considered separate and distinct events even though both VTE and atherosclerosis share common risk factors such as obesity, metabolic syndrome and diabetes mellitus. Venous thrombosis occurs in a low-flow, low-pressure system. Reduced blood flow, activation of coagulation proteins, and insults to the vessel wall are believed to contribute to venous thrombus formation and are not associated with plaque formation in the subendothelial space. By contrast, arterial thrombus occurs in a high-flow, high pressure system. High shear forces are postulated to stress the endothelium stimulating processes conducive to plaque formation, activation of platelets and the coagulation cascade.

Arterial and venous thrombi typically differ in their composition. Venous thrombi are comprised of primarily red cells whereas arterial thrombi predominantly comprise platelets. However, both thrombi contain various degrees of fibrin and cellular elements, including platelets, red cells, and leukocytes.

Platelet activation on the arterial side of the circulation occurs at the site of vascular injury which disrupts the endothelium, exposes procoagulant phospholipids, and leads to the formation of factor Xa and thrombin, which stimulates further platelet activation, thrombin generation and fibrin deposition. Thus, a positive feedback loop is established for further platelet activation and thrombin formation.

In contrast, the role of platelet procoagulation activity on the venous side of the circulation has not been firmly established, but it may be explained by several different mechanisms. First, venous wall injury exposes subendothelial collagen and von Willebrand factor (vWF) which binds to GpIIb receptors localized on the platelet cell membrane and leads to activation and subsequent platelet aggregation. Second, venous stasis due to prolonged immobilization or venous occlusion causes primary activation of coagulation proteins. Thus, differences in pathogenesis may explain why aspirin or anticoagulants may be more effective in preventing thrombi due to vascular injury as opposed to venous stasis.

Even though different mechanisms are responsible for atherosclerosis and venous thromboses, some correlation has been shown. Recent studies have shown that asymptomatic atherosclerosis predisposes individuals to venous thrombosis. Conversely, arterial events were more common in patients with previous idiopathic VTE disease. The strength of this association is greater in patients with idiopathic disease as compared to those with secondary thrombosis. Furthermore, evidence suggests that while arterial and venous thrombogenesis may involve distinct mechanisms, they appear to be nonetheless cooperative, integrated, and reciprocal self-amplifying processes.

Lipoprotein-associated phospholipase A2 (Lp-PLA₂) is a calcium-independent serine lipase that is associated with low-density lipoprotein particles (LDL) in human plasma that is produced predominately by macrophages and lymphocytes. This molecule is associated with key aspects of atherogenesis including: (1) oxidation of phospholipids such as those within oxidized LDL and (2) generation of proinflammatory moieties, such as lysophosphatidylcholine and oxidized free fatty acids. Additionally, these products are involved in recruiting and activating additional monocytes and macrophages. These cells in turn extrude a variety of bioactive substrates into their local environment including cytokines, chemokines, and growth factors that stimulate further inflammation.

Lp-PLA₂ may be a marker for inflammation as studies have shown an independent predictive association between patients with elevated levels of Lp-PLA₂ having no prior history of overt atherosclerotic disease and incidence of cardiovascular events. For example, Lp-PLA₂ levels have been found to be elevated in patients with coronary artery disease and have been associated with increased risk of coronary events in patients with preexisting cardiovascular disease.

Inflammation is thought to be both a cause for, and an outcome of, venous thrombosis and a potent mediator of atherosclerosis and cardiovascular disease. Inflammatory processes increase procoagulant protein levels and enhance tissue factor expression on the surfaces of white blood cells and endothelial cells. These cells, along with endothelial and vascular smooth muscle cells, express or enhance the expression of a variety of bioactive substances including cytokines, chemokines, and adhesion molecules that play a role not only in atherosclerotic plaque progression but also in arterial and venous thrombus development and propagation.

Although Lp-PLA₂ has been associated with pathogenesis at the level of arterial endothelium, association of Lp-PLA₂ with development of VTE as a biological marker has not yet been established.

### EXAMPLE 1

This prophetic example illustrates how the present invention may be used for predicting the development of DVT or recurrence of DVT in patients undergoing anticoagulant therapy for treatment of DVT. Specifically, the following prophetic example is contemplated for those patients who meet the noted clinical case study criteria. That is, the present invention is directed to those patients who do not fall within into the class of patients for which the development of DVT is secondary to one of the noted conditions or predispositions.

A proposed case-control study in which patients with a DVT diagnosed through duplex ultrasonography are enrolled aims to establish a correlation between levels of LP-PLA₂ and the development of idiopathic VTE. The proposed case-control study will involve cases defined based on a lack of compressible venous segment or lack of venous flow in the deep veins of the lower extremities. Patients enrolled in the study will undergo carotid ultrasonography to assess peak systolic and diastolic velocity, degree of stenosis, and presence of plaque in the common, external and internal carotid arteries. The plaque is classified as being any one or the combination of heterogeneous, calcified or irregular.

Patients are classified into primary (idiopathic) or secondary thrombotic categories. The etiology(s) for secondary thrombosis are classified using the following categories: (1) patient related (e.g., age, obesity, smoking status, prolonged immobilization; (2) gastrointestinal (e.g., inflammatory bowel disease); (3) renal (e.g., nephrotic syndrome, chronic renal failure); (4) endocrinological (e.g., diabetes mellitus); (5) hematological (e.g., malignancy); (6) cardiovascular (e.g., hypertension); (7) related to indwelling catheter/devices; (8) drug-related (e.g., chemotherapeutic, oral contraceptives); (9) infection-related (e.g., cellulites); and (10) neurological (e.g., paralysis). Use of lipid lowering drugs (e.g., pravastatin, atorvastatin), aspirin, or clopridogrel are also recorded. The control group consists of patients with no personal or family history of VTE and the patients are matched for age and gender.

Plasma lipoprotein profile will be measured according to standard, validated methods such as those recognized by the Clinical Diagnostic Division Beckman Coulter, Inc., Brea, Calif. Blood from the enrolled patients will be sampled into different anticoagulants and plain tubes for harvesting plasma and serum. Anticoagulants such as EDTA, or heparin, are preferably used in the proposed case study, however any anticoagulant useful in the practice of the present invention may be employed. Lp-PLA₂ level is assessed by a dual monoclonal antibody immunoassay (ELISA) standardized to recombinant Lp-PLA₂ such as the PLAC test produced by diaDexus, Inc, of South San Francisco, Calif.

Patients will be enrolled in the case study if they meet certain, inclusionary criteria. Specifically, patients to be enrolled will have (1) symptomatic vein thrombosis and (2) DVT confirmed by duplex ultrasonography. On the other hand, patients excluded from the present study include those that meet any of the following criteria: (1) prior history of VTE, (2) prior history of ischemic stroke, transient ischemic attack (TIA), acute myocardial infarction, or angina, (3) prior history of peripheral vascular disease or intermittent claudication, (4) prior history of carotid endarterctomy, coronary artery bypass or angioplasty, or leg artery bypass or angioplasty, (5) prior history of thrombophilic conditions (i.e., factor V Leiden, prothrombin 20210A, protein C, deficiency, protein S deficiency, hyperhomocysteinemia, and/or antiphospholipid antibody (lupus anticoagulant, DRVVT, anticardiolipin and/or anti-β₂ glycoprotien I antibodies), (6) prior history of trauma, or (7) prior history of surgery within the previous four weeks.

The concentration of Lp-PLA₂ levels in patients with idiopathic and secondary venous thrombosis with and without atherosclerotic disease will be measured. It is predicted that patients with idiopathic DVT or idiopathic DVT and atherosclerotic disease will have higher concentration of Lp-PLA₂ compared to the secondary thrombosis group. Using a cut-off point of 235 ng/ml (50^{th} percentile for the population) for the serum or plasma Lp-PLA₂ mass concentration, it is predicted that persons with Lp-PLA₂ values in the second and third tertile have a higher risk of VTE events compared to those in the lowest tertile. Further, it is proposed that Lp-PLA₂ concentrations in patients with idiopathic DVT and atherosclerosis will be significantly associated with a greater risk of VTE events in person in the top vs. lowest tertile compared to those with secondary DVT.

Lp-PLA₂ level will be measured using any one of many known and readily available methods. For example, diaDexus, Inc., South San Francisco, Calif., provides a sandwich enzyme immunoassay that uses two highly specific monoclonal antibodies for direct measurements of Lp-PLA₂ in human plasma and serum. The diaDexus method is the only assay that is currently FDA-cleared for use in the United States.

The principle of the test is described in the PLAC test package insert Lp-PLA₂ ELISA test kit generation 3, i.e., PLAC test, diaDexus, Inc, South San Francisco, Calif., which is incorporated herein by reference.

For the variable with continuous scale such as Lp-PLA₂ concentration, we assume that the population means of Lp-PLA₂ concentration for the defined DVT cases and controls are M₁ and M₀, respectively, with the (common) standard deviation, σ, of the measures in their respective normally (or approximately normally) distributed populations. For the determination of effect size, we use the approach defining a standardized mean difference, D, as follows:

D=|M₁−M₀|/σ i.

Once D is defined and given that a two-tailed test is of interest, a total of 252 cases and 252 controls, and a Type-I error rate of 5%, we will be able to calculate the statistical power for various mean comparisons. For example, if the hypotheses of interest are as follows:

H₀:|M₁−M₀|=0

H₁:|M₁−M₀|≠0

A study by Khuseyinova reported in 2005 established that the estimated mean±standard deviation of Lp-PLA₂ was 296.1±122.5 ng/ml for 312 coronary artery disease patients and 266.0±109.8 ng/ml for the 479 controls. Accordingly, the present proposed method of detecting VTE will employ these values. Specifically, the following values will be utilized in conducting the proposed study: M₁=296 ng/ml, M₀=266 ng/ml. Further, the common within-population standard deviation σ=120 ng/ml. Accordingly, the following relationship is derived:

D=|M₁−M₀|/σ=|296−266|/120=0.25

The above recited relationship implies that the means differ by 25% of a standard deviation. Therefore, in order to detect this effect size, the calculated statistical power will be 80%, given that the Type-I error equals 5%, and the sample size is 252 for each group.

Most of the previous studies reported concentrations of Lp-PLA2 as means (and their standard deviations), with or without log transformation. In the present study, we propose comparing mean Lp-PLA2 values, with and without log transformation, between the defined DVT cases and controls, using Student's t-test for two independent samples. In addition, analyses of covariance will be performed to examine the difference in means of Lp-PLA2 between cases and controls, with adjustment for age, gender, smoking, blood pressure, lipids, and body mass index. In terms of risk assessment, we will obtain the odds ratios for DVT, the associated 95% confidence intervals and p-values, according to the quartile values of Lp-PLA2, using the unconditional logistic regression modeling. Subjects in the first quartile of Lp-PLA2 will be used as the reference group for comparison. Furthermore, the multivariate logistic regression modeling will be performed to obtain the odds ratios for DVT, the associated 95% confidence intervals and p-values, according to the quartile values of Lp-PLA2, with adjustment for age, gender, smoking, blood pressure, lipids, and body mass index. A trend test may be conducted with the measurement of Lp-PLA2 treated as the continuous variable, and a p-value of <0.05 is claimed as statistically significant. All data analyses are performed using Statistical Analysis System (SAS).

The relevant data points to be determined in conducting the proposed case study are (1) Lp-PLA2, (2) D-dimer, (3) age, (4) body mass index (BMI), (5) smoking status, (6) gender, (7) total cholesterol, (8) LDL, (9) HDL, and (10) triglycerides.

Levels of Lp-PLA₂ are affected by lipid/cholesterol levels, age, and gender. Other factors may affect Lp-PLA₂ levels but their impact is currently unknown. Accordingly, the impact of these factors on LP-PLA2 may be further evaluated in patients with VTE.

Accordingly, the data retrieved from the above prophetic example may be used to predict the idiopathic development of DVT or the recurrence thereof. Specifically, it is believed that by determining the levels of Lp-PLA₂ in patients meeting the above-noted clinical criteria, a correlation may be drawn between the measured Lp-PLA₂ level and the idiopathic development of DVT.

Although the present invention has been described in detail with reference to a preferred embodiment thereof, other versions or variations thereto are possible. Therefore the spirit and scope of the appended claims should not be limited to the description and the preferred versions contain within this specification.

What is claimed is:

1. A method for predicting the development of deep vein thrombosis in humans, the method comprising
a) drawing a blood sample from a patient, wherein the patient meets a predetermined clinical criteria;
b) analyzing the blood sample to determine a concentration of Lp-PLA₂;
c) comparing the detected concentration of Lp-PLA₂ to a pre-determined reference level;
d) determining whether the patient is likely to develop deep vein thrombosis based on the comparing of the detected concentration to the known standard.

2. The method of claim 1, wherein the predetermined clinical criteria constitutes patients for which the development of deep vein thrombosis is idiopathic.

3. The method of claim 1, wherein the determining step further comprises a direct diagnosis of deep vein thrombosis when the Lp-PLA₂ concentration is at least some pre-determined value.

4. The method of claim 1, wherein the analyzing step comprises utilizing an assay for detecting the concentration of Lp-PLA₂.

5. The method of claim 4, wherein the assay is a dual monoclonal antibody immunoassay.

6. The method of claim 5, wherein the dual monoclonal antibody immunoassay is an ELISA test.

7. The method of claim 1, wherein the pre-determined reference level is based on Lp-PLA₂ levels existing in patients with atherosclerotic disease.

8. The method of claim 1, wherein a venous thromboemoblism is detected by one of a duplex ultrasound ventilation-perfusion scan and a computed tomography scan.

9. The method of claim 1, wherein the blood sample is selected from the group consisting of serum, EDTA plasma and heparin plasma.

10. A method for predicting risk for recurrent DVT comprising the steps of:
a) drawing a blood sample from a patient receiving anticoagulant treatment for DVT, wherein the patient is at risk for developing recurrent DVT after cessation of the anticoagulant treatment;
b) analyzing the blood sample to detect a concentration of Lp-PLA₂;
c) comparing the detected concentration of Lp-PLA₂ to a pre-determined standard;
d) determining based on the comparing step whether the patient is at risk for developing recurrent DVT.

11. The method of claim 10, wherein the analyzing step utilizes an assay for detecting the concentration of Lp-PLA₂.

12. The method of claim 11, wherein the assay is a dual monoclonal antibody immunoassay.

13. The method of claim 12, wherein the dual monoclonal antibody immunoassay is an ELISA test.

14. The method of claim 10, wherein the blood sample is selected from a group consisting of serum, EDTA plasma, and heparin plasma.

15. The method of claim 10, wherein the step of analyzing the blood sample occurs at the time of discontinuation of the anticoagulant.

16. The method of claim 10, further comprising the step of deciding whether the patient should remain on anticoagulants based on the detected level of Lp-PLA₂.
